(19) United States
(12) Patent Application Publication
(10) Pub. No.: US 2003/0113225 A1
(43) Pub. Date: Jun. 19, 2003

(54) Method and device for eliminating odors and killing mold and bacteria associated with unwanted biologicalgrowth in walk-in and box coolers

(76) Inventors: Larry Richard Hunt, Gainesville, GA (US); James Philip Williams, Grayson, GA (US)

Correspondence Address: LARRY RICHARD HUNT, 5003 FARUBROOK LN., GAINESVILLE, GA, 30507, US

(21) Appl. No.: 09/977,776

(22) Filed: Oct. 15, 2001

(51) Int. Cl. A61L 2/00

(52) U.S. Cl. 422/24

(57) ABSTRACT

The present invention provides a method for the treatment of hazardous biologicals present in walk-in and box coolers. Said biologicals unintentionally grow in and cause spoilage of stored goods kept in a cool dark location such as walk-in and box coolers. For example commercial or privet cooler storage facilities. The method eliminates odors and kills bacteria associated with offensive odors and product spoilage. These said hazardous biologicals are neutralized when exposed to the Mixture of ozone and ultraviolet radiation.

### BACKGROUND—FIELD OF INVENTION

This invention relates to ultraviolet radiation and ozone, used in the cleaning of water, sewage, and air. Specifically those dealing with the environment of walk-in and box coolers. Bacteria and mold in this environment promotes spoilage and reduces product usability. These said emissions are hazardous to human health and cause a loss in revenue.

Making the cooler environment cleaner is a mater of removing the pollutants from their source. There are chemicals such as chlorine bleach, fungicides and biocides on the market for controlling bacteria and molds, but that's just adding yet another chemical compounds to the cooler atmosphere. This invention kills bacteria, mold and elimnates odor from walk-in and box coolers with out the harsh unnatural chemicals. Therefore reducing hazardous conditions in walk-in and box coolers.

The treatment method is to use ozone in combination with exposure to ultraviolet radiation. Ozone has been used for over sixty years in Europe for treating contaminates. The role of ozone in waste fluid treatment may be classified as both and oxidant and a germicidal compound. At least four distinct recognized applications of ozone are: (1) as a bactericide; (2) as a viricide; (3) as a powerful chemical oxidant; and (4) as a promoter of hydroxyl radicals when combined with ultraviolet radiation.

The potent germinal properties of ozone have been attributed to its high oxidation potential. Research indicates that disinfections by ozone are a direct result of bacterial cell wall disintegration. Ozone has several attributes in the treatment of waste fluids such as odor control, color removal, and iron and manganese removal. Ozone oxidizes inorganic substances completely and rapidly, e.g., sulfides to sulfates, and nitrates to nitrates. Even greater importance lies in Ozone's capability of breaking down complex organic chemicals.

### BACKGROUND—DESCRIPTION OF PRIOR ART

U.S. Pat. No. 6,096,219 Aug. 1, 2000, to Lawrence M. Green, and Michael G. Nickelsen, both of Fla. Developed a method and apparatus for pretreatment of hazardous waste material. Their invention provided a method for pretreatment of hazardous biological and chemical contaminants from a waste fluid stream prior to discharge to a wastewater treatment facility such as a publicly owned water treatment works. Their apparatus treats waste fluid streams with ozone, then exposes the mixture to ultraviolet radiation.

And

U.S. Pat. No. 6,156,192 Dec. 5, 2000, John M. Rummler, of Kittery Point, Me. Waste treatment system, invention which provides a waste treatment process and apparatus in which a high degree of separation is initially provided for separating solid and liquid components. Once the separator separates the solids and liquids, a fire tube combusts the solids with microwaves. The liquids are treated in a liquid treatment system which includes particle filtration/separation, and treatment with ozone and ultraviolet radiation.

As well as

U.S. Pat No. 6,117,324 Sep. 12 2000, Annel K. Greene; Charles S. Knight, both of Seneca, S.C. A system and process for treating animal waste. Treating the wash water runoff from pen cleaning etc. It includes filtration and ozone treatment of the contaminated water through a series of three graduated holding ponds.

These three different inventions have a common goal of sewage clean up, by appling ozone combined with ultraviolet radiation.

U.S. Pat. No. 6,129,849 Yoshikawa Et al. Oct. 10, 2000, Process for accelerating reaction of ozone with a catalyst for the treatment of water by injection of ozone. This claims the removal of harmful matters, odor and color. For wastewater recycling of fish and farm ponds.

And

U.S. Pat No. 5,935,431 Aug. 10 1999, Amos Korin, of 16 Mountain view Dr, Weston, Conn. 06883 A process and apparatus using ultraviolet and ozone for water purifying and for water disinfections of dental unit water supply systems.

And

U.S. Pat. No. 420,720 Feb. 15 2000, Alfredo J. Teran, John R. Derrick, Jr., of Rockledge; Nidal A. Samad, of Merritt Island; W. Todd Willoughby: Igor A. Vassiliev, Both of Cape Canaveral: Louis V. Mangicarpa; Carlos B. Diax, Both of Merritt Island all of Fla.

The ornamental design of an apparatus for the purification of water, using ultraviolet radiation and ozone. Used to clean drinking water.

All three of this group are different inventions with one common goal to clean water by appling ozone combined with ultraviolet radiation.

U.S. Pat. No. 5,972,196 Oct. 26 1999, Oliver J. Murphy; G. Duncan Hitchens, both of Bryan, Tex. Uses electrochemical production of ozone and hydrogen peroxide. Developed to sterilize medical instruments and medical waste. It oxidizes organics found in clean up wastewater, such as laundry and food production.

And

U.S. Pat. No. 4,752,401 Jun. 21, 1988 By Willem J. Bodenstein, Ft. Myers, Fla. Water treatment system for swimming pools and potable water. For the treatment of re-circulated water, either potable water or for bathing, using at least one ozone producing ultraviolet lamp.

And

U.S. Pat. No. 5,256,379 Oct. 26. 1993 By. Anthony DeLoach, 1630 Jewel Ave., Sarasota, Fla. 34240. Apparatus and method for removing hydrocarbons from airstreams. An apparatus and method for removing hydrocarbon contaminates flowing into a chamber in the presence of a nozzle spraying an atomized reagent such as hydrogen peroxide and/or ozone through the air stream. For the clean up of contaminated soil or ground water. To flush out gasoline contaminations.

All three of this group are yet different inventions with a common use for ozone combined with ultraviolet radiation, to sterilize and remove impurities.

### BACKGROUND—DESCRIPTION OF PRIOR ART

### CONCLUSION

In conclusion, not one of these prior arts addressed the hazardous conditions in walk-in or box coolers. Insofar as we aware, no mold and bacteria growth controller has formerly been developed with the combination and application presented in this patent.

### SUMMARY

This invention is a device that eliminates walk-in and box cooler odors, while killing bacteria and mold contaminants. The device (FIG. 1) houses a combination unit that produces ultraviolet radiation and ozone. The ultraviolet lamp (FIG. 1 part 13) is contained in the one inch elongated tube (FIG. 1 part 19). This tube has a 1×8 inch discharge opening (FIG. 2 part 21) out its side. This discharge opening allows ultraviolet rays and ozone to discharge on to the coolers evaporation coil surface and disinfect the incoming air, and discharges treated vapors into the coolers contained air.

This unit uses the coolers own evaporator fan as the mode of transport for the activated bacteria and mold killing forces. The placement of this invention is critical to its proper operation. It is to be located on the side cabinet wall of the evaporator cabinet. The U.V. light, (FIG. 2-13) and protective steel tube (FIG. 2-19), are located behind the evaporator cooling coil. The coolers evaporator fan draws in air that's confined in the cooler box. This air is pulled across the evaporator coil, Thus removing the heat from said air. The same said fan draws air into the elongated tube, where it is exposed to ultraviolet radiation and ozone concentration. This air continues to flow into the evaporator housing and mixes with the air confined to the cooler box.

The radiation from the ultraviolet light (FIG. 2-13)emission kills the bacteria and breaks down the undesirable carbon molecules. This process eliminates most of the odors, bacteria, and mold thus eliminating them from the air contained in the cooler box. Thereby preventing the accidental discharge contaminants being distributed into the occupied spaces.

The results is a more pleasant and healthier environment. It is well known that ultraviolet light will kill bacteria, and ozone will eliminate odors. It is believed that the combination presented in this application herein, has not been attempted in the past.

### THE ONE CLAIM AND ADVANTAGES

The one claim is the disinfecting qualities of ultraviolet and ozone. The internal treatment of the walk-in and box coolers disinfects the contained air, evaporator cooler coil, condensation pan and the water that's being purged from the system.

This product has been developed, tested and proven its ability to eliminate odors and kill contaminates such as, bacteria, viruses, mold and fungi in walk-in and box coolers. Thus providing a safer and healthier environment for building occupants.

### FIVE FIGURES EXPLAINED

In the drawings, related figs. have the same numbers.

FIG. 1 shows various aspects of a view of the total assembly apparatus for eliminating odors and killing bacteria associated with walk-in and box coolers.

FIG. 2 shows left side view of the electrical containment box, lamp holding section with ballast power pack, ultraviolet lamp and reflecting shield.

FIG. 3 shows a right side view of the apparatus with timer, electrical containment box, ballast and reflecting shield.

FIG. 4 shows a left side profile the same as FIG. 2 with out the reflecting shield.

FIG. 5 shows right side profile same as FIG. 3 with out the reflection shield.

### REFERENCE NUMBERS IN PARTS DRAWINGS EXPLAINED

This parts list depicts the all model Parts list

### Walk-In and Box Cooler Device

11. Florescent lamp ballast, U.V. Ballast 14 Watt.

11-1. Light that shows Florescent lamp is working properly

11-2. 18-gauge wire to lamp plug adapter, 18 Gauge 4 conductor electrical cord 2 feet long it comes with the Ballast.

12. Lamp plug adapter, UV Bulb electrical receiver. It comes with the (no. 1) 14 Watt UV Ballast, Wire and Bulb Assembly.

13. Florescent lamp 14 Watt U.V. Lamp bulb 8 to 12 inches long and ½ inch in diameter

14. Cover for electrical wiring housing Carlon no. E989RRX (6×6)

14-1. Wiring cover from the side showing the ozone sensor penetration

14. Ozone electronic sensor board, Turns off at 0.05 ppm/50 ppb back on at 35 ppb (parts per billion)

15-1. Ozone sensor penetration

16. Ozone sensor board transformer

17. Electrical housing, Carlon no. E987R 6 inch×6 inch×4 inch deep.

18. Stainless steel reflector shield 12 inch by 8 inch flat stock

19. 1 inch steel tube (bulb housing) 12 inch long

20. 1 inch tubing cap

21. Opening cut 1 inch by 8 inch in one side of the in 1 inch steel tubing only.

22. Hole cut 2¼ inch by 1¼ inch in the side of the electrical housing to mount 120-volt receptacle outlet

23. Hole cut 2¼ inch by 1¼ inch in the side of the electrical housing to mount 120-volt receptacle outlet

24. 1 inch pressure connection to mount 1 inch steel tubing to mounting box no. 17

24-1. 1 inch hole cut in back of electrical/mounting box for 1 inch pressure connection

25. Cover switch to cut off the power to the unit when the cover is removed. Cut of switch, hinge lever. 3 amp part # D2F-F1 Mouser electronics 653-D2F-F1

26. Main male power plug, on 16 Gauge Extension Cord With Ground NO. 045131440349

26-1. 16-gauge main power cord, 16 Gauge Extension Cord With Ground NO. 045131440349

27. Male power plug for time clock operation

27-1. 18-gauge time clock power cord woods no. 0600W

28. Water tight electrical cord connector, ½ inch base ⅜ Cord Grip. Appleton Electric CG1850 catalog number. Cable range. 187-312

29. Water tight electrical cord connector, ⅜ Stress reliefs in gray HJ1013GPK25

30. Water tight electrical cord connector, ⅜ Stress relief in gray HJ1013GPK25

31. Fuse housing, 5A Fuse holder 5×20 mm 2701238

32. Time Clock Switch Intermatic Time-All model no. TN811

33. Duplex receptacle Eagle 15 Amp decorator no. 1107GY

34. Duplex receptacle Eagle 15 Amp decorator no. 1107GY

### DETAILED DESCRIPTION OF THE INVENTION

The process and the apparatus description of similar parts has been indicated in the specification and drawings with the same reference numerals where appropriate. The drawings are not to scale and some sections have been enlarged for clarification purposes. All of the various controlling electrical lines have been left out for clarity.

This apparatus and process being described has the capability of disinfecting the air, and contents of walk-in and box coolers.

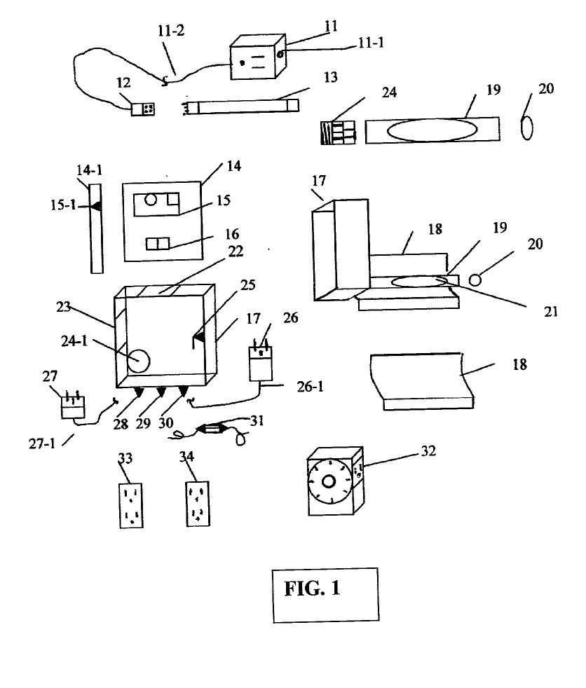

FIG. 1, a drawing describing all components of the device for elimination of odors and killing mold and bacteria associated with unwanted biological growth in walk-in and box coolers.

Specific details are shown as part (11), a 14 watt ballast that supplies the power for (13) ultraviolet lamp. LED light (11-1) gives proof that the bulb is working. Wire lead (11-2) and lamp plug adapter (12) as well as (11-1) and (11) are all part of the Total ballast assembly. (13) 14 watt 8 to 11 inch long ½ inch in diameter ultraviolet c lamp is medical grade lamp with stylizing qualities.

The electrical body consist of (14) the lid for box (17) the lid has mounted on it (15) electronic ozone sensor board and (16) the 12 volt power supply for the sensor. (15-1) a ⅜ inch breather hole in the lid for the sensor to read the ozone it the adjoining space. (17) Electrical housing serves as a wiring box, steel sleeve (19) and steel reflector shield (18) support as well as a mounting device. (19) One inch steel sleeve used to protect the lamp as well as reflect and direct the u.v. rays. (20) is a 1 inch tubing cap it is used to close off the exposed end of (19). (21) is a 1×8 inch notch made on onside of (19) to direct the rays and allow the ozone to be drawn from (13) the encased u.v. lamp.

(21) is a 1 inch hole cut in (17) box for mounting the lamp assembly. (22) and (23) are holes cut 2¼×1¼ inch in the side of the electrical housing (17) for the mounting of (33 and 34) 15 amp duplex receptacle used to power (11) 14 watt ballast and (32) 24 hour adjustable timer. (24) a 1 inch pressure connector used to connect (19) 1 inch steel sleeve to (17) the electrical wiring box. (25) is safety cut off switch mounted in (17) electrical box with contact to (14) box cover, when cover is removed electrical power to the unit is disconnected. (26) and (26-1) are parts of the main power cord assembly consisting of a male plug and 16 gauge 3 conductor wire rubber cord. (27 and 27-1) is part of and 18 gauge power cord assembly 2 conductor wire switch lead for the timer switch. (28, 29, 30) water tight cord connectors. (31) Is a 5-amp fuse holder and fuse used to protect the components in case of a power serge or component shorts.

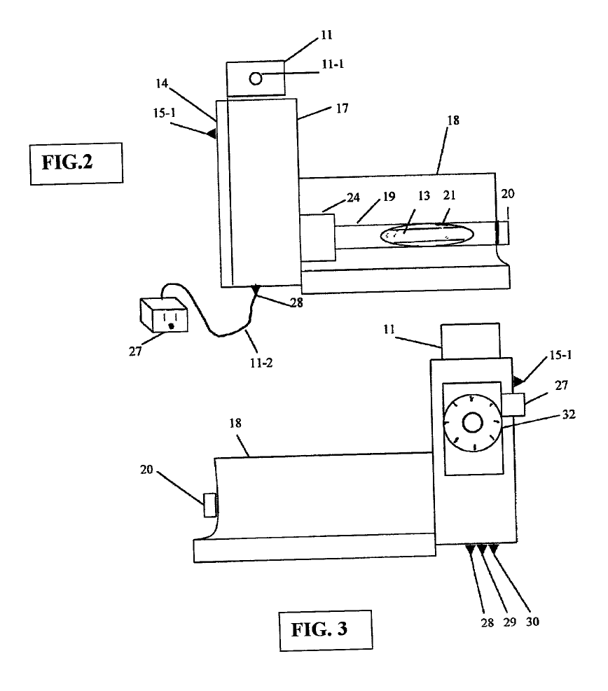

FIG. 2 shows left side view of the electrical containment box (17), lamp holding section (19) with ballast power pack(11), ultraviolet lamp (13) and reflecting shield (18).

FIG. 3 shows a right side view of the apparatus with timer (32), electrical containment box (17), and ballast and reflecting shield (18).

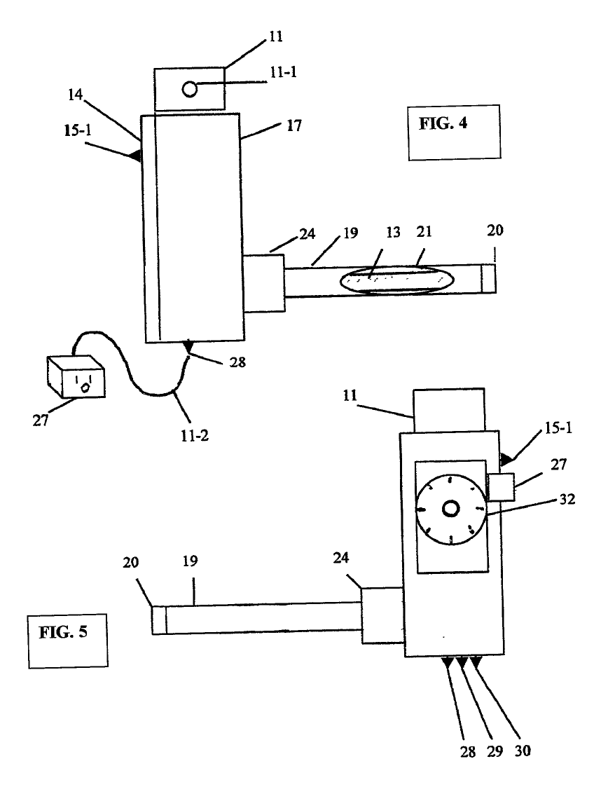

FIG. 4 shows a left side profile the same as FIG. 2 with out the reflecting shield.

FIG. 5 shows right side profile same as FIG. 3 with out the reflection shield.

That completes the parts assembly section.

The one claim and advantages:

1. The one claim is the disinfecting qualities of ultraviolet and ozone. The internal treatment of the walk-in and box coolers disinfects the contained air, evaporator cooler coil, condensation pan and the water that's being purged from the system.
This product has been developed, tested and proven its ability to eliminate odors and kill contaminates such as, bacteria, viruses, mold and fungi in walk-in and box coolers. Thus providing a safer and healthier environment for building occupants.
